(19) United States
(12) Patent Application Publication
(10) Pub. No.: US 2018/0325998 A1
(43) Pub. Date: Nov. 15, 2018

(54) DELIVERABLE EXTRACELLULAR VESICLES INCORPORATING CELL MEMBRANE TRANSPORT PROTEINS

(71) Applicant: Northwestern University, Evanston, IL (US)

(72) Inventors: Joshua N. Leonard, Wilmette, IL (US); Devin M. Stranford, Evanston, IL (US); Michael J. Passineau, Wexford, PA (US)

(73) Assignee: Northwestern University, Evanston, IL (US)

(21) Appl. No.: 15/975,222

(22) Filed: May 9, 2018

Related U.S. Application Data
(60) Provisional application No. 62/503,621, filed on May 9, 2017.

(51) Int. Cl. A61K 38/17; A61K 51/12; A61K 9/127

(52) U.S. Cl. CPC A61K 38/177; A61K 9/127; A61K 51/1234

(57) ABSTRACT

Disclosed are extracellular vesicles (EVs), such as exosomes, comprising a heterologous cell membrane transporter protein, such as the sodium iodide symporter (NIS). Also disclosed are methods of using the disclosed EVs for delivering agents to recipient cells and methods for measuring efficacy of delivery by the EVs to the recipient cells. Also disclosed are method of making the disclosed EVs and cell lines for producing the EVs.

### CROSS-REFERENCE TO RELATED PATENT APPLICATIONS

The present application claims the benefit of priority under 35 U.S.C. 119€ to U.S. Provisional Application No. 62/503,621, filed on May 9, 2017, the content of which is incorporated herein by reference in its entirety.

### STATEMENT REGARDING FEDERALLY SPONSORED RESEARCH OR DEVELOPMENT

This invention was made with government support under DGE1324585 awarded by the National Science Foundation. The government has certain rights in the invention.

### BACKGROUND

The present invention relates to extracellular vesicles that may be utilized as delivery vehicles in molecule systems. In particular, the disclosed subject matter relates to extracellular vesicles that incorporate and deliver membrane transporter proteins, such as the sodium (Na) iodine symporter (NIS) and/or nucleic acid that encodes the NIS, which may be used determining the efficacy of delivery of extracellular vesicle cargo to recipient cells.

Secreted extracellular vesicles, such as exosomes and microvesicles, are nanometer-scale lipid vesicles that are produced by many cell types. Extracellular vesicles are know to transfer proteins, nucleic acids, membrane material, and other molecules between cells in the human body, as well as those of other animals. Targeted exosomes in particular have a wide variety of potential therapeutic uses and have already been shown to be effective for delivery of RNA to neural cells and tumor cells in mice.

Here, we describe extracellular vesicles that may be utilized as delivery vehicles in molecule systems. In particular, the disclosed subject matter relates to extracellular vesicles that incorporate and deliver membrane transporter proteins, such as the sodium (Na) iodine symporter (NIS) and/or nucleic acid that encodes the NIS, which may be used determining the efficacy of delivery of extracellular vesicle cargo to recipient cells. In particular, the disclosed extracellular vesicles may be utilized to deliver the NIS protein or nucleic acid encoding the NIS to recipient cells. After delivery, functional delivery may be assessed by contacting the recipient cells with a labeled substrate for the NIS and detecting and/or measuring uptake of the labeled substrate by the recipient cells.

### SUMMARY

Disclosed are extracellular vesicles (EVs), such as exosomes and microvesicles, comprising a heterologous cell membrane transporter protein, such as the sodium iodide symporter (NIS). Also disclosed are methods of using the disclosed EVs for delivering agents to targets cells and methods for measuring efficacy of delivery by the EVs to the target cells. Also disclosed are method of making the disclosed EVs and cell lines for producing the EVs.

### BRIEF DESCRIPTION OF THE FIGURES

FIG. 1. Schematic illustration of the production of extracellular vesicles (EVs) including microvesicles and exosomes from an EV producer cell and uptake of the EVs by an EV recipient cell.

FIG. 2. Schematic illustration of an EV comprising a membrane surface ligand corresponding to a receptor present on the cell surface of an EV recipient cell.

FIG. 3. Enhancement of EV uptake by treatment with polybrene and spinoculation. (A) Illustration of experimental design. (B) Uptake of CD63-GFP-labeled EVs by MCF-7 cells or MDA-MB-231 cells was quantified following incubation for 2 h under the delivery conditions indicated (for conditions including spinoculation, spinoculation was performed for 1 h and then cells were returned to the incubator for an additional 1 h before analysis). Experiments were performed in biological triplicate, and error bars indicate one standard deviation. Statistical tests comprise two-tailed Student's t-tests (*p<0.05). Results are representative of two independent experiments.

FIG. 4. Dependence of EV uptake on cell growth matrix stiffness. (A) Illustration of experimental design. (B) Gelatin matrix stiffness quantified by rheology (no cells added). (C) CD63-GFP-labeled EV uptake by MCF-7 cells or MDA-MB-231 cells during culture on various substrates following 2 h of incubation. Experiments were performed in biological triplicate, and error bars indicate one standard deviation. Statistical tests comprise paired two-tailed Student's t-tests, individually comparing each gelatin concentration condition to TCPS (*p<0.05). Results are representative of two independent experiments. TCPS, tissue culture polystyrene.

FIG. 5. Schematic illustration of analysis of fluorescent cargo present in EVs after uptake by EV recipient cells at time point 1 (4 hours) and 2 (12 hours). Dashed line illustrates expected fluorescence based on the half-life of the fluorophore dTomato (˜25 h) and the cell doubling time (˜38 h) versus the solid line which illustrates the observed fluorescence.

FIG. 6. Schematic representation of incorporation of the sodium (Na) iodide symporter (NIS) protein or NIS mRNA into EVs in EV producer cell and uptake of EVs by EV recipient cell and presentation and/or expression of NIS on the surface of the EV recipient cell. A labeled substrate for NIS (e.g., Tc-99m pertechnetate) can be transported by the NIS into the EV recipient cell to demonstrate functional uptake of the EV cargo.

FIG. 7. Expression of NIS in EV recipient cells after uptake of NIS microvesicles but not NIS exosomes.

FIG. 8. Loading of NIS into EVs and delivery of NIS to HPAECs. (A) Cell lysates (protein mass indicated) or EVs (2.9×10⁸ per lane) derived from either HEK293FT or DS4 cells were loaded and NIS was detected by anti-FLAG antibodies. (B) HPAECs were incubated with EVs (1×10¹⁰ EVs delivered per 5×10⁴ cells) for 16 h, and then lysates from cells were loaded (10 μg protein per lane) and NIS was detected by anti-FLAG antibodies.

FIG. 9. Functional evaluation of NIS delivered to HPAECs via EVs. (A,B,C) HPAECs were incubated with EVs (1×10¹⁰ EVs delivered per 5×10⁴ cells) for 16 h, incubated with the radioisotope Tc-99m pertechnetate for 1 h, washed, and then imaged via SPECT. Tc-99m uptake was evidenced in representative images (A) and quantified via analysis of SPECT data (B). DS3 and DS4 cells served as positive controls by constitutively expressing untagged NIS (DS3) or the same NIS-FLAG that was loaded into EVs (DS4). This experiment was conducted in biological triplicate, and error bars indicate one standard deviation.

FIG. 10. Mechanisms and barriers to EV-mediated delivery of biomolecules to HPAECs. This cartoon summarizes our proposed conceptual model of EV-mediated delivery of NIS to HPAECs. Key features include (i) both exosomes and microvesicles incorporate NIS protein and deliver that protein to recipient cells (HPAECs); (ii) exosomes are degraded in recipient cells without experiencing membrane fusion; (iii) microvesicles mediate functional delivery of NIS to recipient cells by membrane fusion at the cell surface and/or in endosomal compartments; (iv) membrane fusion between microvesicles and recipient cell membrane(s) results in functional delivery of NIS protein and/or NIS mRNA, the latter of which may be translated in recipient cells.

### DETAILED DESCRIPTION

The present invention is described herein using several definitions, as set forth below and throughout the application.

Unless otherwise specified or indicated by context, the terms “a”, “an”, and “the” mean “one or more.” For example, “a protein,” “ligand,” and “receptor” should be interpreted to mean “one or more proteins,” “one or more ligands,” and “one or more receptors,” respectively.

As used herein, “about,” “approximately,” “substantially,” and “significantly” will be understood by persons of ordinary skill in the art and will vary to some extent on the context in which they are used. If there are uses of these terms which are not clear to persons of ordinary skill in the art given the context in which they are used, “about” and “approximately” will mean plus or minus ≤10% of the particular term and “substantially” and “significantly” will mean plus or minus >10% of the particular term.

As used herein, the terms “include” and “including” have the same meaning as the terms “comprise” and “comprising” in that these latter terms are “open” transitional terms that do not limit claims only to the recited elements succeeding these transitional terms. The term “consisting of,” while encompassed by the term “comprising,” should be interpreted as a “closed” transitional term that limits claims only to the recited elements succeeding this transitional term. The term “consisting essentially of,” while encompassed by the term “comprising,” should be interpreted as a “partially closed” transitional term which permits additional elements succeeding this transitional term, but only if those additional elements do not materially affect the basic and novel characteristics of the claim.

Disclosed are extracellular vesicles. The term “extracellular vesicles” should be interpreted to include all nanometer-scale lipid vesicles that are secreted and/or budding by cells such as exosomes and microvesicles, respectively.

Extracellular vesicles may be obtained from so-called extracellular vesicle (EV) producer cells. Extracellular vesicles may be taken up by so-called extracellular vesicle (EV) recipient cells. As utilized herein, the term “recipient cell” may be interchangeably with the term “target cell.”

The disclosed extracellular vesicles may comprise proteins, polypeptides, or peptides. As used herein, the terms “protein” or “polypeptide” or “peptide” may be used interchangeable to refer to a polymer of amino acids. Typically, a “polypeptide” or “protein” is defined as a longer polymer of amino acids, of a length typically of greater than 50, 60, 70, 80, 90, or 100 amino acids. A “peptide” is defined as a short polymer of amino acids, of a length typically of 50, 40, 30, 20 or less amino acids.

A “protein” as contemplated herein typically comprises a polymer of naturally or non-naturally occurring amino acids (e.g., alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine). The proteins contemplated herein may be further modified in vitro or in vivo to include non-amino acid moieties. These modifications may include but are not limited to acylation (e.g., O-acylation (esters), N-acylation (amides), S-acylation (thioesters)), acetylation (e.g., the addition of an acetyl group, either at the N-terminus of the protein or at lysine residues), formylation lipoylation (e.g., attachment of a lipoate, a C8 functional group), myristoylation (e.g., attachment of myristate, a C14 saturated acid), palmitoylation (e.g., attachment of palmitate, a C16 saturated acid), alkylation (e.g., the addition of an alkyl group, such as an methyl at a lysine or arginine residue), isoprenylation or prenylation (e.g., the addition of an isoprenoid group such as farnesol or geranylgeraniol), amidation at C-terminus, glycosylation (e.g., the addition of a glycosyl group to either asparagine, hydroxylysine, serine, or threonine, resulting in a glycoprotein). Distinct from glycation, which is regarded as a nonenzymatic attachment of sugars, polysialylation (e.g., the addition of polysialic acid), glypiation (e.g., glycosylphosphatidylinositol (GPI) anchor formation, hydroxylation, iodination (e.g., of thyroid hormones), and phosphorylation (e.g., the addition of a phosphate group, usually to serine, tyrosine, threonine or histidine).

The term “amino acid residue” also may include amino acid residues contained in the group consisting of homocysteine, 2-Aminoadipic acid, N-Ethylasparagine, 3-Aminoadipic acid, Hydroxylysine, β-alanine, β-Amino-propionic acid, allo-Hydroxylysine acid, 2-Aminobutyric acid, 3-Hydroxyproline, 4-Aminobutyric acid, 4-Hydroxyproline, piperidinic acid, 6-Aminocaproic acid, Isodesmosine, 2-Aminoheptanoic acid, allo-Isoleucine, 2-Aminoisobutyric acid, N-Methylglycine, sarcosine, 3-Aminoisobutyric acid, N-Methylisoleucine, 2-Aminopimelic acid, 6-N-Methyllysine, 2,4-Diaminobutyric acid, N-Methylvaline, Desmosine, Norvaline, 2,2′-Diaminopimelic acid, Norleucine, 2,3-Diaminopropionic acid, Ornithine, and N-Ethylglycine.

The proteins disclosed herein may include “wild type” proteins and variants, mutants, and derivatives thereof. As used herein the term “wild type” is a term of the art understood by skilled persons and means the typical form of an organism, strain, gene or characteristic as it occurs in nature as distinguished from mutant or variant forms. As used herein, a “variant, “mutant,” or “derivative” refers to a protein molecule having an amino acid sequence that differs from a reference protein or polypeptide molecule. A variant or mutant may have one or more insertions, deletions, or substitutions of an amino acid residue relative to a reference molecule. A variant or mutant may include a fragment of a reference molecule. For example, a mutant or variant molecule may one or more insertions, deletions, or substitution of at least one amino acid residue relative to a reference polypeptide (e.g., SEQ ID NO:1).

Regarding proteins, a “deletion” refers to a change in the amino acid sequence that results in the absence of one or more amino acid residues. A deletion removes at least 1, 2, 3, 4, 5, 10, 20, 50, 100, or 200 amino acids residues or a range of amino acid residues bounded by any of these values (e.g., a deletion of 5-10 amino acids). A deletion may include an internal deletion or a terminal deletion (e.g., an N-terminal truncation or a C-terminal truncation of a reference polypeptide). A “variant,” “mutant,” or “derivative” of a reference polypeptide sequence may include a deletion relative to the reference polypeptide sequence.

Regarding proteins, “fragment” is a portion of an amino acid sequence which is identical in sequence to but shorter in length than a reference sequence. A fragment may comprise up to the entire length of the reference sequence, minus at least one amino acid residue. For example, a fragment may comprise from 5 to 1000 contiguous amino acid residues of a reference polypeptide, respectively. In some embodiments, a fragment may comprise at least 5, 10, 15, 20, 25, 30, 40, 50, 60, 70, 80, 90, 100, 150, 250, or 500 contiguous amino acid residues of a reference polypeptide; in other embodiments, a fragment may comprise less than about 5, 10, 15, 20, 25, 30, 40, 50, 60, 70, 80, 90, 100, 150, 250, or 500 contiguous amino acid residues of a reference polypeptide; or in other embodiments, a fragment has a length within a range bounded by any of these values (e.g., a range of 50-100 contiguous amino acids of a reference polypeptide). Fragments may be preferentially selected from certain regions of a molecule. The term “at least a fragment” encompasses the full length polypeptide. For example, a fragment of a protein may comprise or consist essentially of a contiguous portion of an amino acid sequence of the full-length proteins of SEQ ID NO:1. A fragment may include an N-terminal truncation, a C-terminal truncation, or both truncations relative to the full-length protein. A “variant,” “mutant,” or “derivative” of a reference polypeptide sequence may include a fragment of the reference polypeptide sequence.

Regarding proteins, the words “insertion” and “addition” refer to changes in an amino acid sequence resulting in the addition of one or more amino acid residues. An insertion or addition may refer to 1, 2, 3, 4, 5, 10, 20, 30, 40, 50, 60, 70, 80, 90, 100, 150, 200, or more amino acid residues, or a range of amino acid residues bounded by any of these values (e.g., an insertion or addition of 5-10 amino acids). A “variant,” “mutant,” or “derivative” of a reference polypeptide sequence may include an insertion or addition relative to the reference polypeptide sequence. A variant of a protein may have N-terminal insertions, C-terminal insertions, internal insertions, or any combination of N-terminal insertions, C-terminal insertions, and internal insertions.

A “fusion polypeptide” refers to a polypeptide comprising at the N-terminus, the C-terminus, or at both termini of its amino acid sequence a heterologous amino acid sequence. A “variant” of a reference polypeptide sequence may include a fusion polypeptide comprising the reference polypeptide.

Regarding proteins, the phrases “percent identity” and “% identity,” refer to the percentage of residue matches between at least two amino acid sequences aligned using a standardized algorithm. Methods of amino acid sequence alignment are well-known. Some alignment methods take into account conservative amino acid substitutions. Such conservative substitutions, explained in more detail below, generally preserve the charge and hydrophobicity at the site of substitution, thus preserving the structure (and therefore function) of the polypeptide. Percent identity for amino acid sequences may be determined as understood in the art. (See, e.g., U.S. Pat. No. 7,396,664, which is incorporated herein by reference in its entirety). A suite of commonly used and freely available sequence comparison algorithms is provided by the National Center for Biotechnology Information (NCBI) Basic Local Alignment Search Tool (BLAST), which is available from several sources, including the NCBI, Bethesda, Md., at its website. The BLAST software suite includes various sequence analysis programs including “blastp,” that is used to align a known amino acid sequence with other amino acids sequences from a variety of databases. As described herein, variants, mutants, or fragments (e.g., a protein variant, mutant, or fragment thereof) may have 99%, 98%, 97%, 96%, 95%, 94%, 93%, 92%, 91%, 90%, 80%, 70%, 60%, 50%, 40%, 30%, or 20% amino acid sequence identity relative to a reference molecule (e.g., relative to SEQ ID NO:1).

Regarding proteins, percent identity may be measured over the length of an entire defined polypeptide sequence, for example, as defined by a particular SEQ ID number, or may be measured over a shorter length, for example, over the length of a fragment taken from a larger, defined polypeptide sequence, for instance, a fragment of at least 15, at least 20, at least 30, at least 40, at least 50, at least 70 or at least 150 contiguous residues. Such lengths are exemplary only, and it is understood that any fragment length supported by the sequences shown herein, in the tables, figures or Sequence Listing, may be used to describe a length over which percentage identity may be measured.

Regarding proteins, the amino acid sequences of variants, mutants, or derivatives as contemplated herein may include conservative amino acid substitutions relative to a reference amino acid sequence. For example, a variant, mutant, or derivative protein may include conservative amino acid substitutions relative to a reference molecule. “Conservative amino acid substitutions” are those substitutions that are a substitution of an amino acid for a different amino acid where the substitution is predicted to interfere least with the properties of the reference polypeptide. In other words, conservative amino acid substitutions substantially conserve the structure and the function of the reference polypeptide. The following table provides a list of exemplary conservative amino acid substitutions which are contemplated herein:

| Original Residue | Conservative Substitution |
|---|---|
| Ala | Gly, Ser |
| Arg | His, Lys |
| Asn | Asp, Gln, His |
| Asp | Asn, Glu |
| Cys | Ala, Ser |
| Gln | Asn, Glu, His |
| Glu | Asp, Gln, His |
| Gly | Ala |
| His | Asn, Arg, Gln, Glu |
| Ile | Leu, Val |
| Leu | Ile, Val |
| Lys | Arg, Gln, Glu |
| Met | Leu, Ile |
| Phe | His, Met, Leu, Trp, Tyr |
| Ser | Cys, Thr |
| Thr | Ser, Val |
| Trp | Phe, Tyr |
| Tyr | His, Phe, Trp |
| Val | Ile, Leu, Tyr |

Conservative amino acid substitutions generally maintain (a) the structure of the polypeptide backbone in the area of the substitution, for example, as a beta sheet or alpha helical conformation, (b) the charge or hydrophobicity of the molecule at the site of the substitution, and/or (c) the bulk of the side chain. Non-conservative amino acid substitutions generally do not maintain (a) the structure of the polypeptide backbone in the area of the substitution, for example, as a beta sheet or alpha helical conformation, (b) the charge or hydrophobicity of the molecule at the site of the substitution, and/or (c) the bulk of the side chain.

The disclosed proteins, mutants, variants, or described herein may have one or more functional or biological activities exhibited by a reference polypeptide (e.g., one or more functional or biological activities exhibited by wild-type protein). For example, the disclosed proteins, mutants, variants, or derivatives thereof may have one or more biological activities that include sodium iodide symporter activity (which may include transport of substrates, which optionally are labeled substrates, such as radioactive iodide and/or radioactive technetate (e.g., ^{99m}Tc-pertechnetate).

The disclosed proteins may be substantially isolated or purified. The term “substantially isolated or purified” refers to proteins that are removed from their natural environment, and are at least 60% free, preferably at least 75% free, and more preferably at least 90% free, even more preferably at least 95% free from other components with which they are naturally associated.

Also disclosed herein are polynucleotides, for example polynucleotide sequences that encode proteins (e.g., DNA and/or RNA such as mRNA that encode a polypeptide having the amino acid sequence of SEQ ID NO: 1 or a polypeptide variant having an amino acid sequence with at least about 20%, 30%, 40%, 50%, 60%, 70%, 80%, 90%, 95%, 96%, 97%, 98%, or 99% sequence identity to SEQ ID NO: 1.

The terms “polynucleotide,” “polynucleotide sequence,” “nucleic acid” and “nucleic acid sequence” refer to a nucleotide, oligonucleotide, polynucleotide (which terms may be used interchangeably), or any fragment thereof. These phrases also refer to DNA or RNA (e.g., mRNA) of genomic, natural, or synthetic origin (which may be single-stranded or double-stranded and may represent the sense or the antisense strand).

Regarding polynucleotide sequences, the terms “percent identity” and “% identity” refer to the percentage of residue matches between at least two polynucleotide sequences aligned using a standardized algorithm. Such an algorithm may insert, in a standardized and reproducible way, gaps in the sequences being compared in order to optimize alignment between two sequences, and therefore achieve a more meaningful comparison of the two sequences. Percent identity for a nucleic acid sequence may be determined as understood in the art. (See, e.g., U.S. Pat. No. 7,396,664, which is incorporated herein by reference in its entirety). A suite of commonly used and freely available sequence comparison algorithms is provided by the National Center for Biotechnology Information (NCBI) Basic Local Alignment Search Tool (BLAST), which is available from several sources, including the NCBI, Bethesda, Md., at its website. The BLAST software suite includes various sequence analysis programs including “blastn,” that is used to align a known polynucleotide sequence with other polynucleotide sequences from a variety of databases. Also available is a tool called “BLAST 2 Sequences” that is used for direct pairwise comparison of two nucleotide sequences. “BLAST 2 Sequences” can be accessed and used interactively at the NCBI website. The “BLAST 2 Sequences” tool can be used for both blastn and blastp (discussed above).

Regarding polynucleotide sequences, percent identity may be measured over the length of an entire defined polynucleotide sequence, for example, as defined by a particular SEQ ID number, or may be measured over a shorter length, for example, over the length of a fragment taken from a larger, defined sequence, for instance, a fragment of at least 20, at least 30, at least 40, at least 50, at least 70, at least 100, or at least 200 contiguous nucleotides. Such lengths are exemplary only, and it is understood that any fragment length supported by the sequences shown herein, in the tables, figures, or Sequence Listing, may be used to describe a length over which percentage identity may be measured.

Regarding polynucleotide sequences, “variant,” “mutant,” or “derivative” may be defined as a nucleic acid sequence having at least 50% sequence identity to the particular nucleic acid sequence over a certain length of one of the nucleic acid sequences using blastn with the “BLAST 2 Sequences” tool available at the National Center for Biotechnology Information's website. (See Tatiana A. Tatusova, Thomas L. Madden (1999), “Blast 2 sequences—a new tool for comparing protein and nucleotide sequences”, FEMS Microbiol Lett. 174:247-250). Such a pair of nucleic acids may show, for example, at least 60%, at least 70%, at least 80%, at least 85%, at least 90%, at least 91%, at least 92%, at least 93%, at least 94%, at least 95%, at least 96%, at least 97%, at least 98%, or at least 99% or greater sequence identity over a certain defined length.

Nucleic acid sequences that do not show a high degree of identity may nevertheless encode similar amino acid sequences due to the degeneracy of the genetic code where multiple codons may encode for a single amino acid. It is understood that changes in a nucleic acid sequence can be made using this degeneracy to produce multiple nucleic acid sequences that all encode substantially the same protein. For example, polynucleotide sequences as contemplated herein may encode a protein and may be codon-optimized for expression in a particular host. In the art, codon usage frequency tables have been prepared for a number of host organisms including humans, mouse, rat, pig, E. coli, plants, and other host cells.

A “recombinant nucleic acid” is a sequence that is not naturally occurring or has a sequence that is made by an artificial combination of two or more otherwise separated segments of sequence. This artificial combination is often accomplished by chemical synthesis or, more commonly, by the artificial manipulation of isolated segments of nucleic acids, e.g., by genetic engineering techniques known in the art. The term recombinant includes nucleic acids that have been altered solely by addition, substitution, or deletion of a portion of the nucleic acid. Frequently, a recombinant nucleic acid may include a nucleic acid sequence operably linked to a promoter sequence. Such a recombinant nucleic acid may be part of a vector that is used, for example, to transform a cell.

The nucleic acids disclosed herein may be “substantially isolated or purified.” The term “substantially isolated or purified” refers to a nucleic acid that is removed from its natural environment, and is at least 60% free, preferably at least 75% free, and more preferably at least 90% free, even more preferably at least 95% free from other components with which it is naturally associated.

“Transformation” or “transfected” describes a process by which exogenous nucleic acid (e.g., DNA or RNA) is introduced into a recipient cell. Transformation or transfection may occur under natural or artificial conditions according to various methods well known in the art, and may rely on any known method for the insertion of foreign nucleic acid sequences into a prokaryotic or eukaryotic host cell. The method for transformation or transfection is selected based on the type of host cell being transformed and may include, but is not limited to, bacteriophage or viral infection or non-viral delivery. Methods of non-viral delivery of nucleic acids include lipofection, nucleofection, microinjection, electroporation, heat shock, particle bombardment, biolistics, virosomes, liposomes, immunoliposomes, polycation or lipid:nucleic acid conjugates, naked DNA, artificial virions, and agent-enhanced uptake of DNA. Lipofection is described in e.g., U.S. Pat. Nos. 5,049,386, 4,946,787; and 4,897,355) and lipofection reagents are sold commercially (e.g., Transfectam™ and Lipofectin™) Cationic and neutral lipids that are suitable for efficient receptor-recognition lipofection of polynucleotides include those of Feigner, WO 91/17424; WO 91/16024. Delivery can be to cells (e.g. in vitro or ex vivo administration) or target tissues (e.g. in vivo administration). The term “transformed cells” or “transfected cells” includes stably transformed or transfected cells in which the inserted DNA is capable of replication either as an autonomously replicating plasmid or as part of the host chromosome, as well as transiently transformed or transfected cells which express the inserted DNA or RNA for limited periods of time.

The polynucleotide sequences contemplated herein may be present in expression vectors. For example, the vectors may comprise: (a) a polynucleotide encoding an ORF of a protein (such as NIS having an amino acid sequence of SEQ ID NO:1 or a variant thereof) The polynucleotide present in the vector may be operably linked to a prokaryotic or eukaryotic promoter. “Operably linked” refers to the situation in which a first nucleic acid sequence is placed in a functional relationship with a second nucleic acid sequence. For instance, a promoter is operably linked to a coding sequence if the promoter affects the transcription or expression of the coding sequence. Operably linked DNA sequences may be in close proximity or contiguous and, where necessary to join two protein coding regions, in the same reading frame. Vectors contemplated herein may comprise a heterologous promoter (e.g., a eukaryotic or prokaryotic promoter) operably linked to a polynucleotide that encodes a protein. A “heterologous promoter” refers to a promoter that is not the native or endogenous promoter for the protein or RNA that is being expressed.

As used herein, “expression” refers to the process by which a polynucleotide is transcribed from a DNA template (such as into and mRNA or other RNA transcript) and/or the process by which a transcribed mRNA is subsequently translated into peptides, polypeptides, or proteins. Transcripts and encoded polypeptides may be collectively referred to as “gene product.” If the polynucleotide is derived from genomic DNA, expression may include splicing of the mRNA in a eukaryotic cell.

The term “vector” refers to some means by which nucleic acid (e.g., DNA) can be introduced into a host organism or host tissue. There are various types of vectors including plasmid vector, bacteriophage vectors, cosmid vectors, bacterial vectors, and viral vectors. As used herein, a “vector” may refer to a recombinant nucleic acid that has been engineered to express a heterologous polypeptide (e.g., the fusion proteins disclosed herein). The recombinant nucleic acid typically includes cis-acting elements for expression of the heterologous polypeptide.

Any of the conventional vectors used for expression in eukaryotic cells may be used for directly introducing DNA into a subject. Expression vectors containing regulatory elements from eukaryotic viruses may be used in eukaryotic expression vectors (e.g., vectors containing SV40, CMV, or retroviral promoters or enhancers). Exemplary vectors include those that express proteins under the direction of such promoters as the SV40 early promoter, SV40 later promoter, metallothionein promoter, human cytomegalovirus promoter, murine mammary tumor virus promoter, and Rous sarcoma virus promoter. Expression vectors as contemplated herein may include eukaryotic or prokaryotic control sequences that modulate expression of a heterologous protein (e.g. the fusion protein disclosed herein). Prokaryotic expression control sequences may include constitutive or inducible promoters (e.g., T3, T7, Lac, trp, or phoA), ribosome binding sites, or transcription terminators.

The vectors contemplated herein may be introduced and propagated in a prokaryote, which may be used to amplify copies of a vector to be introduced into a eukaryotic cell or as an intermediate vector in the production of a vector to be introduced into a eukaryotic cell (e.g. amplifying a plasmid as part of a viral vector packaging system). A prokaryote may be used to amplify copies of a vector and express one or more nucleic acids, such as to provide a source of one or more proteins for delivery to a host cell or host organism. Expression of proteins in prokaryotes may be performed using Escherichia coli with vectors containing constitutive or inducible promoters directing the expression of either a protein or a fusion protein comprising a protein or a fragment thereof. Fusion vectors add a number of amino acids to a protein encoded therein, such as to the amino terminus of the recombinant protein. Such fusion vectors may serve one or more purposes, such as: (i) to increase expression of recombinant protein; (ii) to increase the solubility of the recombinant protein; (iii) to aid in the purification of the recombinant protein by acting as a ligand in affinity purification (e.g., a His tag); (iv) to tag the recombinant protein for identification (e.g., such as Green fluorescence protein (GFP) or an antigen (e.g., HA) that can be recognized by a labelled antibody); (v) to promote localization of the recombinant protein to a specific area of the cell (e.g., where the protein is fused (e.g., at its N-terminus or C-terminus) to a nuclear localization signal (NLS) which may include the NLS of SV40, nucleoplasmin, C-myc, M9 domain of hnRNP A1, or a synthetic NLS). The importance of neutral and acidic amino acids in NLS have been studied. (See Makkerh et al. (1996) Curr Biol 6(8):1025-1027). Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase.

The presently disclosed methods may include delivering one or more polynucleotides, such as or one or more vectors as described herein, one or more transcripts thereof, and/or one or proteins transcribed therefrom, to a host cell. Further contemplated are host cells produced by such methods, and organisms (such as animals, plants, or fungi) comprising or produced from such cells. The disclosed extracellular vesicles may be prepared by introducing vectors that express mRNA encoding a fusion protein and a cargo RNA as disclosed herein. Conventional viral and non-viral based gene transfer methods can be used to introduce nucleic acids in mammalian cells or target tissues. Non-viral vector delivery systems include DNA plasmids, RNA (e.g. a transcript of a vector described herein), naked nucleic acid, and nucleic acid complexed with a delivery vehicle, such as a liposome. Viral vector delivery systems include DNA and RNA viruses, which have either episomal or integrated genomes after delivery to the cell.

In the methods contemplated herein, a host cell may be transiently or non-transiently transfected (i.e., stably transfected) with one or more vectors described herein. In some embodiments, a cell is transfected as it naturally occurs in a subject (i.e., in situ). In some embodiments, a cell that is transfected is taken from a subject (i.e., explanted). In some embodiments, the cell is derived from cells taken from a subject, such as a cell line. Suitable cells may include stem cells (e.g., embryonic stem cells and pluripotent stem cells). A cell transfected with one or more vectors described herein may be used to establish a new cell line comprising one or more vector-derived sequences. In the methods contemplated herein, a cell may be transiently transfected with the components of a system as described herein (such as by transient transfection of one or more vectors, or transfection with RNA), and modified through the activity of a complex, in order to establish a new cell line comprising cells containing the modification but lacking any other exogenous sequence.

Deliverable Extracellular Vesicles Incorporating Cell Membrane Transporter Proteins

The presently disclosed subject matter relates to extracellular vesicles (EVs) that comprise a cell membrane transporter protein or nucleic acid encoding the cell membrane transporter protein. The cell membrane transporter protein functions in a cell to facilitate, either actively (e.g., via ATP exchange) or passively, the transport of ions or cations across the cell membrane. The cell membrane transporter protein may be heterologous in regard to an EV-producer cell (e.g., where the EV-producer cell is transfected, either stably or transiently with a vector that expresses a cell membrane transporter protein not naturally expressed in the EV-producer cell).

Suitable cell membrane transporter proteins for use in the disclosed subject matter may include, but are not limited to, the sodium iodide symporter (NIS). As such, the disclosed EVs may comprise the NIS protein and/or nucleic acid encoding the NIS protein.

The NIS protein has been cloned and is known in the art. (See Smanik et al., “Cloning of the human sodium iodide symporter,” Biochem. Biophys. Res. Commun. 226 (2), 339-345 (1996); the content of which is incorporated herein by reference in its entirety. The NIS is also known as the sodium/iodide cotransporter or as the solute carrier family 5, member 5 (SLC5A5) that in humans is encoded by the SLC5A5 gene.

The human sodium iodide symporter has 643 amino acids and it sequence is provided under GenBank accession number AAB17378 as:

| (SEQ ID NO: 1) | |
|---|---|
| 1 | meavetgerp tfgawdygvf almllvstgi glwvglargg qrsaedfftg grrlaalpvg |
| 61 | lslsasfmsa vqvlgvpsea yryglkflwm clgqllnsvl tallfmpvfy rlgltstyey |
| 121 | lemrfsravr lcgtlqyiva tmlytgiviy apalilnqvt gldiwaslls tgiictfyta |
| 181 | vggmkavvwt dvfqvvvmls gfwvvlargv mlvggprqvl tlaqnhsrin lmdfnpdprs |
| 241 | rytfwtfvvg gtlvwlsmyg vnqaqvqryv acrtekqakl allinqvglf livssaaccg |
| 301 | ivmfvfytdc dplllgrisa pdqympllvl difedlpgvp glflacaysg tlstastsin |
| 361 | amaavtvedl ikprlrslap rklviiskgl sliygsaclt vaalssllgg gvlqgsftvm |
| 421 | gvisgpllga filgmflpac ntpgvlaglg aglalslwva lgatlyppse qtmrvlpssa |
| 481 | arcvalsvna sglldpallp andssrapss gmdasrpala dsfyaisyly ygalgtlttv |
| 541 | lcgalisclt gptkrstlap gllwwdlarq tasvapkeev ailddnlvkg peelptgnkk |
| 601 | ppgflptned rlfflgqkel egagswtpcv ghdggrdqqe tnl |

The NIS has been characterized molecularly. (See Darrouzet et al., “The sodium/iodide symporter: State of the art of its molecular characterization,” Biochimica et Biophysica Acta 1838 (2014): 244-253; the content of which is incorporated herein by reference in its entirety). The NIS is a transmembrane glycoprotein with a molecular weight of 87 kDa and 13 transmembrane domains, which transports two sodium cations (Na⁺) for each iodide anion (I⁻) into the cell. NIS mediated uptake of iodide into follicular cells of the thyroid gland is the first step in the synthesis of thyroid hormone.^{[9]} In the NIS, the N-terminus extremity is extracellular and the C-terminus is intracellular. The intracellular C-terminus contains 100 amino acids and represents the longest stretch of amino acid residues predicted to lie outside the membrane. This portion contains numerous potential phosphorylation sites (PKA, PKC, CKII), two of which have been biochemically validated in rat NIS (T575 and S581). This domain also bears several potential binding sites for regulatory proteins and is predicted by bioinformatics to have few secondary structures, and thus to be intrinsically unstructured.

The disclosed extracellular vesicles may include, but are not limited to exosomes and microvesicles. Microvesicles are known in the art and typically are larger than exosomes having an average effective diameter of 100-1000 nm versus 10-100 nm for exosomes. In addition, microvesicles are non-homogeneous vesicles generated by outward budding and shedding from the cell membrane of an EV producer cell. Exosomes are more homogeneous and are generated by the inward budding of endosomal membranes within larger intracellular multivesicular bodies (MVBs) of an EV producer cell. The fusion of these intracellular MVBs containing the exosomes with the cell membrane results in the release of the contained exosomes into the extracellular milieu.

The disclosed extracellular vesicles typically include a cell membrane transporter protein or nucleic acid encoding the cell membrane transporter protein. The disclosed extracellular vesicles may include a protein or polypeptide having the amino acid sequence of SEQ ID NO:1 or having an amino acid that is at least about 50%, 60%, 70%, 80%, 90%, 95%, 96%, 97%, 98%, or 99% identical to the amino acid sequence of SEQ ID NO:1, wherein the protein or polypeptide preferably exhibits one or more biological activities of the sodium iodide symporter. In some embodiments, the disclosed extracellular vesicles may include nucleic acid encoding a protein or polypeptide having the amino acid sequence of SEQ ID NO:1 or having an amino acid that is at least about 50%, 60%, 70%, 80%, 90%, 95%, 96%, 97%, 98%, or 99% identical to the amino acid sequence of SEQ ID NO:1, wherein the protein or polypeptide preferably exhibits one or more biological activities of the sodium iodide symporter.

The disclosed extracellular vesicles optionally may include additional components. In some embodiments, the extracellular vesicles further comprise a surface ligand that binds to receptor on a recipient cell. In other embodiments, the extracellular vesicles further comprise a therapeutic agent for delivery to a recipient cell. (See, e.g., U.S. Publication No. 20170087987; the content of which is incorporated herein by reference in its entirety).

The disclosed extracellular vesicles may be utilized in methods for delivering cargo to recipient cells. The disclosed methods may include: (a) contacting extracellular vesicles with recipient cells, the extracellular vesicles comprising a cell membrane transporter protein or nucleic acid encoding the cell membrane transporter protein (optionally where the cell membrane transporter is heterologous to the EV-producer cell); optionally (b) contacting the recipient cells with a labeled substrate for the heterologous cell membrane transporter protein; and optionally (c) detecting uptake of the labeled substrate in the recipient cells. Suitable cell membrane transporter proteins may include, but are not limited to the sodium iodide symporter (NIS) protein. Suitable labeled substrates for the NIS may include but are not limited to radioactive iodide or radioactive technetate (e.g., ^{99m}Tc-pertechnetate).

In some embodiments of the disclosed methods, the recipient cells may be inoculated with the extracellular vesicles by performing inoculation in the presence of centrifugation otherwise referred to as “spinoculation.” In some embodiments, after adding the extracellular vesicles to a culture of recipient cells, the culture of recipient cells may be spinoculated at a centrifugal force of at least about 50, 100, 200, 300, 400, or 500 g (or at a centrifugal force bounded by any of these values such as 100-500 g) for at least about 10, 20, 30, 60, or 120 minutes (or for a time within a range bounded by any of these values such as 60-120 minutes). In some embodiments, the extracellular vesicles may be added to the recipient cells at a multiplicity of inoculation (MOI) of at least about 10², 10³, 10⁴, 10⁵, 10⁶, 10⁷, or 10⁸ extracellular vesicles per recipient cells (or within a MOI range bounded by any of these values such as 10⁵-10⁸).

In some embodiments of the disclosed methods, the extracellular vesicles may be treated with a cationic reagent prior to inoculating recipient cells with the extracellular vesicles. Cationic reagents may include, but are not limited to, cationic polymers, and preferably non-toxic, biodegradable cationic polymers. In some embodiments of the disclosed methods, the extracellular vesicles may be treated with cationic polymers selected from, but not limited to, polybrene, polyethyleneimine, polylysin, polyornithine, amine-containing cyclodextrin derivatives, chitosan, histone polymers, collagen, and amine-containing dendrimers

The disclosed extracellular vesicles may be prepared by methods disclosed in the art. (See, e.g., methods disclosed in the following Examples and associated citations). In some embodiments, the disclosed extracellular vesicles may be prepared by expressing in an EV-producing cell line a cell membrane transport protein (e.g., the sodium iodide symporter (NIS) protein) and isolating extracellular vesicles comprising the cell membrane transport protein or comprising nucleic acid encoding the cell membrane transport protein. In some embodiments, the cationic polymer may be added to a suspension of extracellular vesicles at a concentration of at least about 0.5, 1.0, 2.0, 3.0, 4.0, 5.0, 6.0, 7.0, 8.0, 9.0, 10.0, 20.0, 50.0 μg/mL (or within a concentration range bounded by any of these values such as 2.0-10.0 μg/mL).

In the disclosed methods, after contacting recipient cells with extracellular vesicles comprising the NIS or comprising mRNA encoding the NIS and permitting uptake of the extracellular vesicles by the recipient cells, the recipient cells may be contacted with a substrate for the NIS, for example, by adding the substrate to a culture of the recipient cells. The substrate may be a labeled substrate whose uptake by the recipient cells can be detected and measured in order to assess uptake of the extracellular vesicles and the extracellular vesicles' cargo by the recipient cells. Substrates for the NIS are known in the art. (See, e.g., (See Darrouzet et al., “The sodium/iodide symporter: State of the art of its molecular characterization,” Biochimica et Biophysica Acta 1838 (2014): 244-253; the content of which is incorporated herein by reference in its entirety). Suitable substrates may include radiolabeled substrates or otherwise labeled substrates. Suitable substrates may include optionally radiolabeled anions (or cations) transported by the NIS such as I⁻, ClO₃⁻, SCN⁻, SeCN⁻, NO₃⁻, Br⁻, ClO₄⁻, ReO₄⁻, TcO4⁻ (e.g., ^{99m}Tc-pertechnetate).

Also disclosed herein are cells that may be utilized to produce the disclosed extracellular vesicles. The disclosed cells may include recombinant EV-producing cell lines into which a nucleic acid that encodes a cell membrane transport protein has been introduced, optionally where the cell membrane transport protein is heterologous and optionally where the cell membrane transport protein is the sodium iodide symporter (NIS).

### ILLUSTRATIVE EMBODIMENTS

The following embodiments are illustrative and should not be interpreted to limit the scope of the claimed subject matter.

### Embodiment 1

Extracellular vesicles comprising a heterologous cell membrane transporter protein and/or an mRNA encoding a heterologous cell membrane transporter protein, optionally wherein the extracellular vesicles are exosomes or microvesicles.

### Embodiment 2

The extracellular vesicles of embodiment 1, wherein the heterologous cell membrane transporter protein is the sodium iodide symporter (NIS), optionally having an amino sequence of SEQ ID NO:1 or an amino acid sequence that is at least about 80% identical to SEQ ID NO:1, wherein the heterologous cell membrane transporter protein exhibits iodide transport activity.

### Embodiment 3

The extracellular vesicles of embodiment 1 or 2, further comprising a surface ligand that binds to receptor on a recipient/target cell for the extracellular vesicles.

### Embodiment 4

The extracellular vesicles of any of the foregoing embodiments, further comprising a therapeutic agent for delivery to a target cell.

### Embodiment 5

A method comprising contacting the extracellular vesicles of any of the foregoing embodiments with recipient/target cells and delivering the heterologous cell membrane transporter protein and/or the mRNA encoding the heterologous cell membrane transporter protein to the recipient/target cells, preferably in a manner in which the recipient/target cells uptake the extracellular vesicles and after uptaking the extracellular vesicles the recipient/target cells comprise and/or express the heterologous cell membrane transporter protein.

### Embodiment 6

The method of embodiment 5, wherein prior to contacting the extracellular vesicles with the recipient/target cells, the extracellular vesicles are treated with a cationic agent.

### Embodiment 7

The method of embodiment 5 or 6, wherein the recipient/target cells are contacted with the extracellular vesicles under centrifugal force (i.e., via spinoculation).

### Embodiment 8

The method of any of embodiments 5-7, wherein the heterologous cell membrane transporter protein is NIS and the method further comprises treating the recipient/target cells with a labeled substrate for NIS that is transported by NIS across the cell membrane and measuring uptake of the labeled substrate by the recipient/target cells.

### Embodiment 9

The method of embodiment 8, wherein the labeled substrate is radioactive iodine or radioactive technetate (e.g., ^{99m}Tc-pertechnetate).

### Embodiment 10

A method for preparing any of the extracellular vesicles of embodiments 1-4, the method comprising expressing the heterologous cell membrane transporter protein in an EV-producing cell line.

### Embodiment 11

The method of embodiment 10, wherein the heterologous cell membrane transporter protein is NIS.

### Embodiment 12

The method of embodiment 10 or 11, wherein expressing the heterologous cell membrane transporter protein in the EV-producing cell line comprises introducing into the EV-producing cell line a nucleic acid that encodes the heterologous cell membrane transporter protein.

### Embodiment 13

A recombinant EV-producing cell line into which a nucleic acid that encodes a heterologous cell membrane transporter protein has been introduced.

### EXAMPLES

The following Examples are illustrative and are not intended to limit the scope of the claimed subject matter.

### Example 1—A Systematic Evaluation of Factors Affecting Extracellular Vesicle Uptake and Functional Cargo Delivery

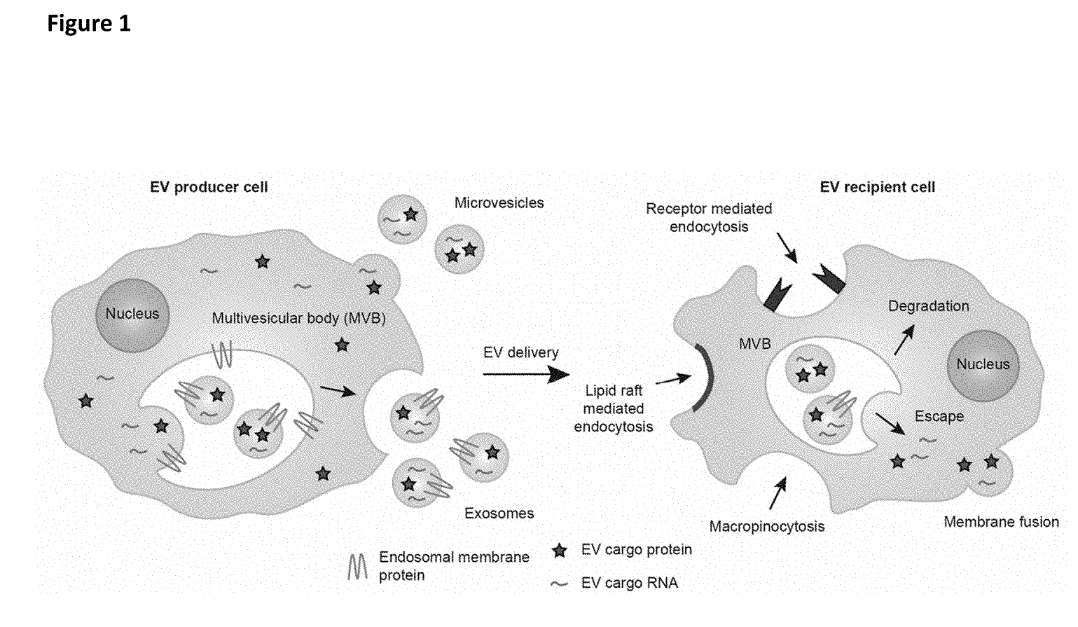

Eukaryotic cells naturally release into the extracellular milieu a heterogeneous population of membrane particles referred to as “extracellular vesicles” (EVs). (See FIG. 1). These so-called EVs are classified based on their size and their mode of release from the cell, among other distinguishing characteristics, as “microvesicles” or “exosomes.” (See FIG. 1). Microvesicles are larger than exosomes having an average effective diameter of 100-1000 nm versus 10-100 nm for exosomes. In addition, microvesicles are non-homogeneous vesicles generated by outward budding and shedding from the cell membrane. (See FIG. 1). In contrast, exosomes represent a more homogeneous population of vesicles that are generated by the inward budding of endosomal membranes within larger intracellular multivesicular bodies (MVBs). (See FIG. 1). The fusion of these intracellular MVBs containing the exosomes with the cell membrane results in the release of the contained exosomes into the extracellular milieu. (See FIG. 1). EVs then can be taken up by a recipient cell by processes including receptor mediated endocytosis (e.g., when the EVs have a ligand on the surface corresponding to a respective receptor on the recipient cell), lipid raft mediated endocytosis, micropinocytosis, and membrane fusion.

As such, EVs have the natural capacity to transfer biological material from one donor cell to another recipient cell. Biological material transferred by EVs may include membrane material, cytoplasmic material (e.g., proteins and RNA), and genetic material. Accordingly, EVs have been utilized as therapeutic vehicles for the delivery of bioactive proteins, lipids, and nucleic acids.

However, the use of EVs as therapeutic vehicles presents several challenges. First, in order for EVs to efficiently delivery a therapeutic cargo, the therapeutic cargo should be specifically loaded into the EVs. The most common approach is treating the EVs with electroporation in the presence of the therapeutic cargo, but such electroporation can produce cargo aggregates that are hard to separate from EVs and may not be effectively delivered to a recipient cell.

Another approach involves cargo loading during biogenesis of the EVs. This approach requires that the cargo be present or expressed in the EV-producer cell and relies on mass action for cargo loading. The loading mechanisms in this approach are still largely not understood.

The goal in these approaches for cargo loading is achieving efficient cargo loading into EVs, efficient EV uptake by recipient cells and delivery of the EV cargo. However, even when cargo is efficiently loading into EVs, the cargo may be degraded prior to being delivered to a recipient cell, and/or the EV may not be efficiently taken up by the recipient cell. Therefore, methods for unambiguously evaluating functional delivery of EV cargo are needed.

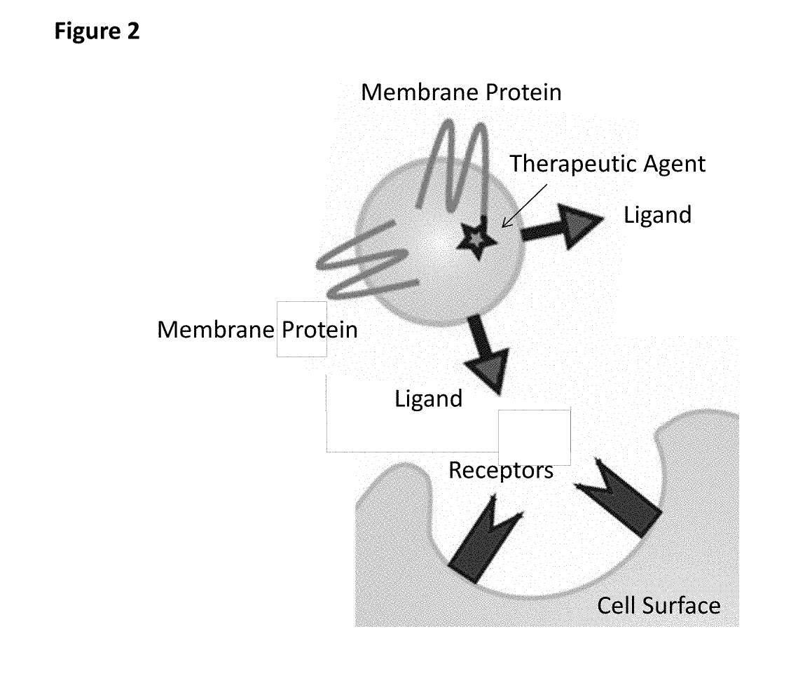

A targeting peptide may be expressed in an EV-producer cell in order to produce EVs having the targeting peptide on their membrane surface. (See FIG. 2 and Stranford et al., “A Systematic Evaluation of Factors Affecting Extracellular Vesicle Uptake by Breast Cancer Cells,” Tissue Eng Part A. 2017 November; 23(21-22):1274-1282 (hereinafter “Stranford 2017”); the content of which is incorporated herein by reference in its entirety). Targeting peptides have been observed to improve EV uptake by a recipient cell expressing a receptor for the targeting peptides. (See Stranford 2017″).

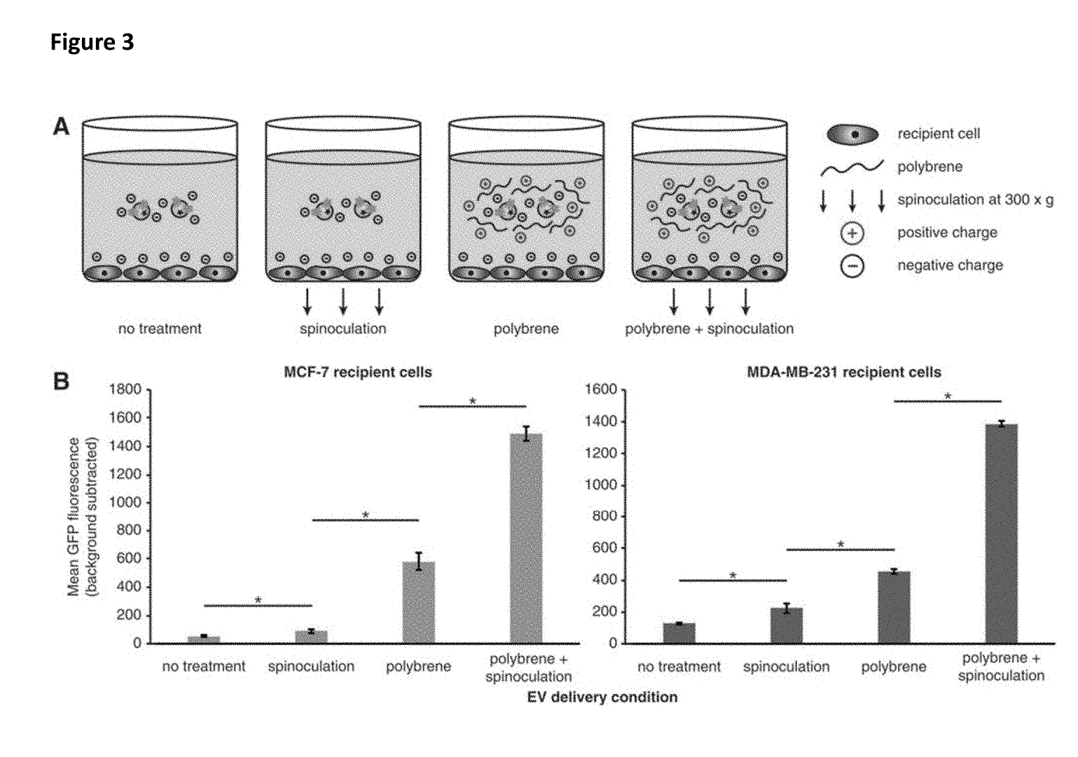

In addition, because the membrane surface of EVs and the cell membrane surface of recipient cells generally are negatively charged, prior to inoculating the recipient cells with the EVs, the EVs may be treated with a cationic reagent (e.g., a cationic polymer such as polybrene). Treatment of EVs with a cationic reagent prior to inoculating recipient cells with the EVs has been observed to improve EV uptake by the recipient cells. (See FIG. 3 and Stranford 2017). In addition, recipient cells may be subjected to centrifugal inoculation (i.e., spinoculation) with EVs in order to improve EV uptake. (See FIG. 3 and Stranford 2017).

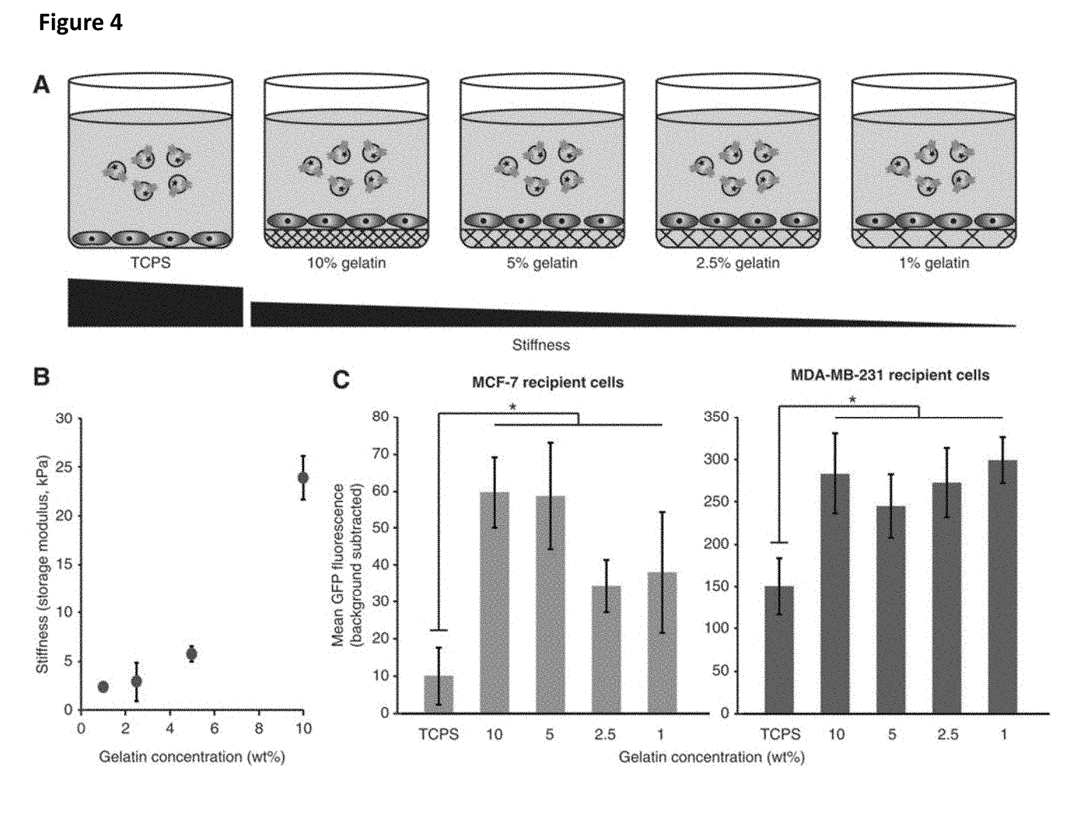

The nature of the growth surface of recipient cells also affects the efficiency of EV uptake by the recipient cells. Recipient cells grown on a “soft matrix” such as a matrix comprising gelatin (1%, 2.5%, 5%, or 10% having a storage moduli of 2.4 kPa, 6 kPa, 12 kPa, and 24 kPa, respectively) versus a “hard matrix” such as tissue culture grade polystyrene (having a storage modulus of ˜3Gpa) take up EVs more efficiently. (See FIG. 4 and Stranford 2017).

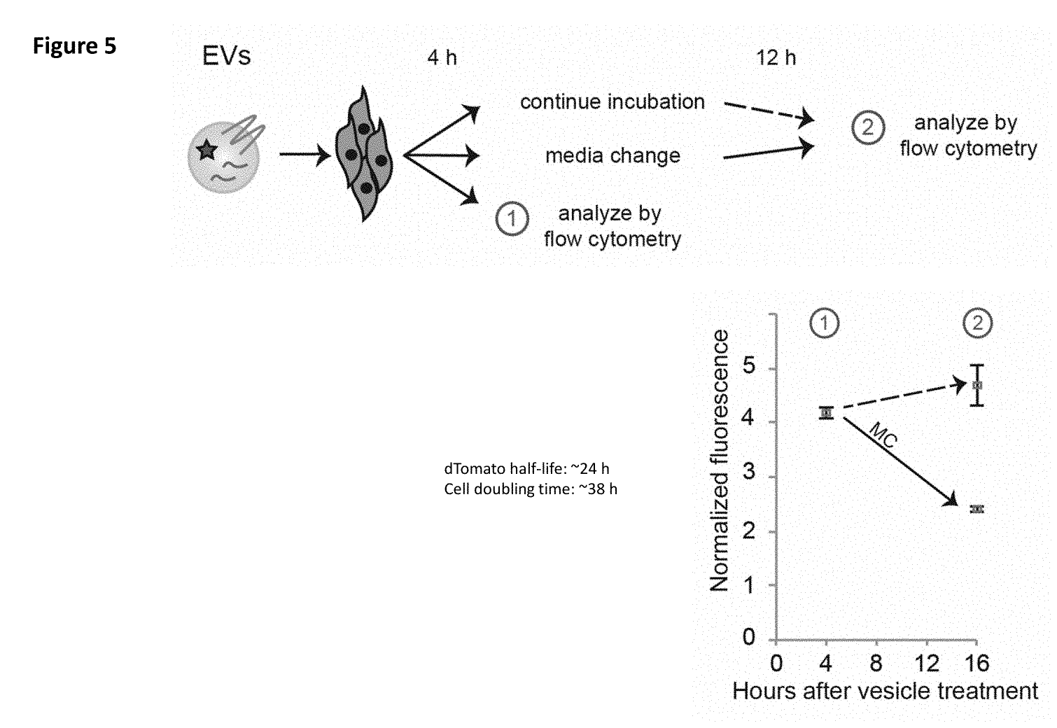

Fluorescent cargo often is utilized to assess EV uptake. However, a general problem in the field of EV study is that much of the cargo that is delivered by EVs is degraded in recipient cells prior to the functional activity of the cargo being assessed. In addition, fluorescent cargo is not ideal for assessing functional delivery because fluorescent cargo can be degraded prior to EV uptake and/or EVs can release fluorescent cargo prior to EV uptake which released fluorescent cargo can enter recipient cells in the absence of EV uptake. (See FIG. 5, and Hung and Leonard, “A platform for actively loading cargo RNA to elucidate limiting steps in EV-mediated delivery,” J. Extracell. Vesicles, 2016; 5:31027; the content of which is incorporated herein by reference in its entirety).

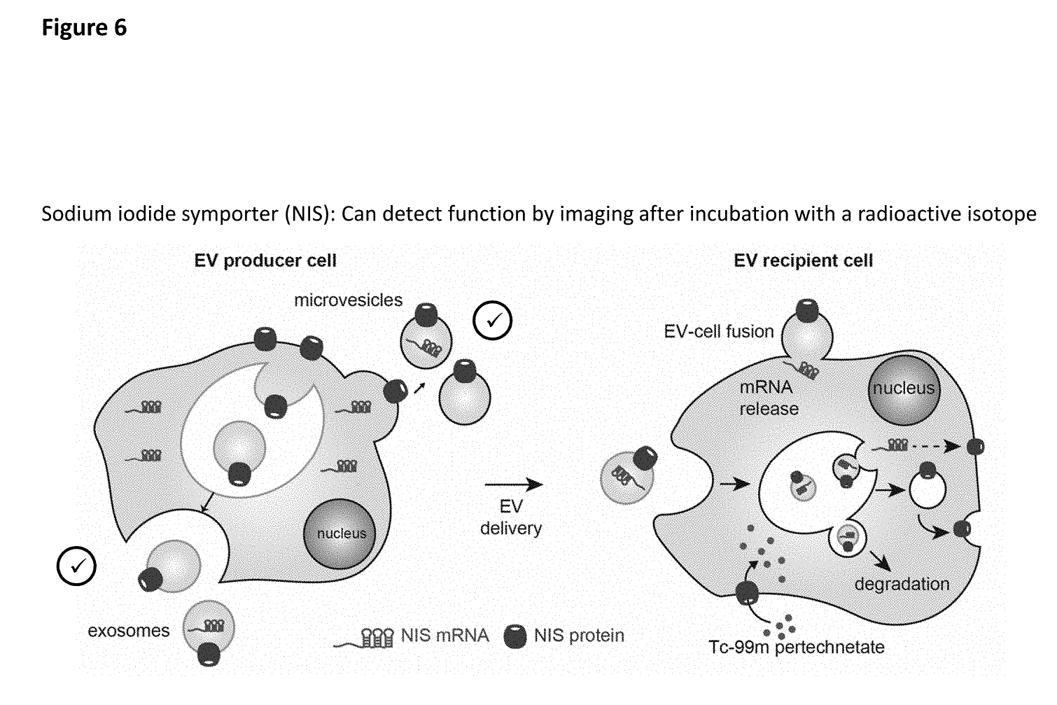

As such, the present inventors have developed a platform to unambiguously evaluate the functional delivery of EV cargo to recipient cells using the sodium (Na) iodide symporter (NIS) as EV cargo. After the NIS is delivered to a recipient cell (e.g., by inoculating the recipient cell with EVs comprising the NIS or an mRNA encoding the NIS, see FIG. 6), function of the NIS in the recipient cell may be assessed by administering a labeled substrate for the NIS (e.g., Tc-99m pertechnetate) to the cellular milieu of the recipient cell and assessing uptake of the labeled substrate by the recipient cell. (See FIG. 6). In order to function as a symporter, the NIS must be properly oriented in the recipient cell membrane after EV uptake.

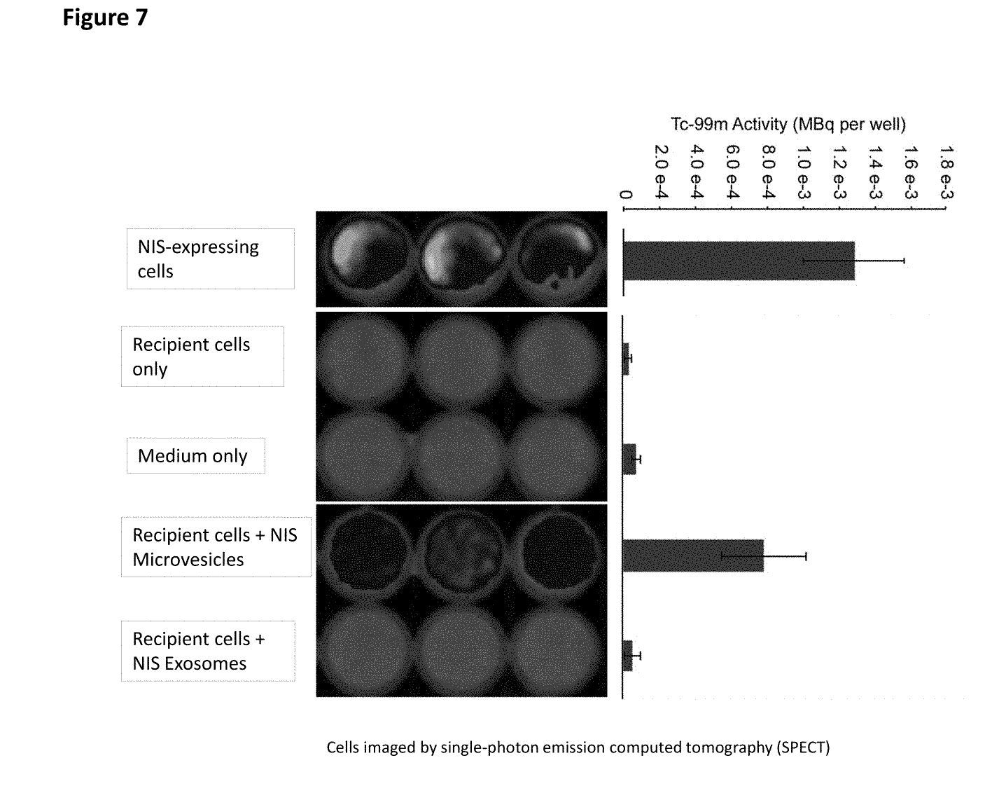

Using such a platform, the present inventors have observed that EVs carrying NIS and/or NIS mRNA as cargo can be used to inoculate recipient cells and produce recipient cells that contain and/or express the NIS, which demonstrated unambiguous evidence of EV-cell membrane fusion. (See FIG. 7). Interestingly, the present inventors have found that microvesicles, but not exosomes, could mediate functional deliver of NIS to recipient cells. (See FIG. 7). Therefore, the present inventors have established a model system for evaluating functional EV-mediated delivery.

### Example 2—Extracellular Vesicles Capable of Incorporating and Delivering the Sodium Iodide Symporter to Recipient Cells

Abstract

Extracellular vesicles (EV) are nanoscale lipid particles secreted by nearly all cell types. They have been shown to transport protein and RNA between cells and serve an important role in intercellular communication. This natural delivery function has made them an attractive platform for delivery of therapeutic molecules. However, achieving functional delivery of these molecules remains a challenge. The sodium iodide symporter (NIS) is a membrane protein that actively transports iodide into cells and can be used in nuclear imaging by transporting radioactive iodine or other isotopes. This invention describes the use of a subset of EVs incorporating NIS to transfer NIS to the membrane of recipient cells, where NIS is functional. These EVs therefore allow for imaging of recipient cells via methods utilizing NIS (e.g., SPECT imaging), which enables one to track EV uptake and evaluate membrane fusion and functional cargo delivery between EVs and recipient cells both in vitro and in vivo.

Applications

Applications for the disclosed subject matter include but are not limited to: (i) evaluating functional EV cargo delivery to recipient cells; (ii) evaluating EV-recipient cell membrane fusion; (iii) evaluating EV localization in vivo (in experimental animals and in humans); and (iv) delivering NIS to cancer cells for radioisotope-mediated diagnosis and treatment.

Advantages

Advantages of the disclosed subject matter include but are not limited to: (i) currently there is no technology that can unambiguously and generally evaluate EV functional delivery in unmodified cell lines or animals; (ii) currently there is no comparable technology for unambiguously evaluating EV fusion with recipient cell membranes; (iii) NIS-based imaging provides a highly sensitive method for detecting EV delivery and localization as compared to fluorescent proteins and dyes; and (iv) NIS-based imaging can be applied to human patients (as well as to experimental animals), unlike imaging via fluorescent labels or bioluminescence.

Prior Art

The disclosed subject matter may be practiced using methods and compositions in the prior art and adapted for use in methods and compositions in the prior art, including but not limited to: Vituret, C. et al., “Transfer of the cystic fibrosis transmembrane conductance regulator to human cystic fibrosis cells mediated by extracellular vesicles.” Hum Gene Ther, 27(2): 166-83 (2016); U.S. Published Application No. 20-15/0093433, “Targeted and Modular Exosome Loading System”; and U.S. Published Application No. 2017/0087087, “Targeted Extracellular Vesicles Comprising Membrane Proteins with Engineered Glycosylation Sites,” the contents of which are incorporated herein by reference in their entireties.

Conclusion

EV delivery is an attractive strategy delivering a wide range of therapeutics. However, there is not currently a generalizable way to track and unambiguously evaluate functional delivery of EV cargo. The technology disclosed here provides a subset of EVs well-suited to this purpose, and it identifies a method for evaluating EV transfer via NIS-based imaging.

### Example 3

Deliverable Extracellular Vesicles Incorporating Sodium Iodide Symporter

Forward

This example summarizes a pilot study, in which we evaluated methods for harnessing EVs to delivery sodium iodide symporters (NIS) to recipient cells.

Summary

Extracellular vesicles (EVs), of which exosomes are the most studied, are nanoscale “packages” that carry molecules between cells (1). Recently, EVs have begun to attract serious attention as potential biological therapeutic agents following their use to deliver siRNA to the brain (2, 3). Compared to synthetic gene delivery vehicles, EVs may exhibit higher stability, lower toxicity and immunogenicity (4), and utilization of native mechanisms for efficient delivery of cargo molecules into the cytoplasm of recipient cells where they are biologically active (1). The overall goal of this pilot study was to explore the feasibility of producing, storing, and transporting EVs to mediate delivery of a reporter gene suitable for use in swine and eventually humans.

In preliminary work, we demonstrated that we can produce, store, and functionally test protein-loaded EVs. Specifically, we found that EVs can be refrigerated or frozen without any loss in functional delivery of a fluorescent protein, while lyophilization substantially reduced EV delivery.

In the work shown here, we evaluated the extent of EV incorporation and functional delivery of the sodium/iodide symporter (NIS) reporter protein. Recent advances in the scientific literature have suggested that EV-mediated delivery of protein is an attractive strategy for pursuing the core goals of this project. In particular, EVs have recently been reported to transfer the integral membrane glycoprotein, cystic fibrosis transmembrane conductance regulator (CFTR) to recipient cells, in such a manner that the transferred protein is inserted into the membrane of recipient cells and is functional (5). This recent evidence that CFTR, a transmembrane protein, can functionally insert into the membranes of cells transduced by EVs raises the intriguing possibility that other proteins might be delivered using such an approach. In light of this emergent advancement in EV-mediated protein therapy, our proposal to deliver the integral membrane protein sodium/iodide symporter (NIS) as a reporter gene (6) becomes all the more exciting.

We successfully generated these key results: (i) NIS can be loaded into two distinct subsets of EVs: (1) microvesicles (which bud directly from the cell membrane) and exosomes (which originate from endosomal compartment); (ii) Both subsets EVs evaluated (microvesicles and exosomes) are efficiently taken up by clinically-relevant recipient cells (primary human pulmonary artery endothelial cells, HPAECs); (iii) Microvesicles successfully confer functional delivery of the NIS reporter protein to recipient cells (HPAECs), while exosomes confer NIS delivery that is non-functional; and (iv) Altogether, EVs meet key criteria for a plausible next-generation biopharmaceutical agent.

Key Findings

We evaluated whether two clinically-relevant subsets of EVs incorporate and functionally deliver the porcine sodium/iodide symporter (NIS) to clinically relevant primary (cadaver-derived) human pulmonary artery endothelial cells (HPAECs). Note that due to the mechanism of the NIS reporter protein, which spans the plasma membrane and acts as a transporter, functional delivery of NIS to recipient cells is expected to require that (i) NIS protein is functionally installed in the plasma membrane of the recipient cells (e.g., via fusion between the EV membrane and the recipient cell membrane), and (ii) such installation must place NIS in the proper orientation (i.e., such that normally extracellular face of NIS indeed faces the exterior of the recipient cell).

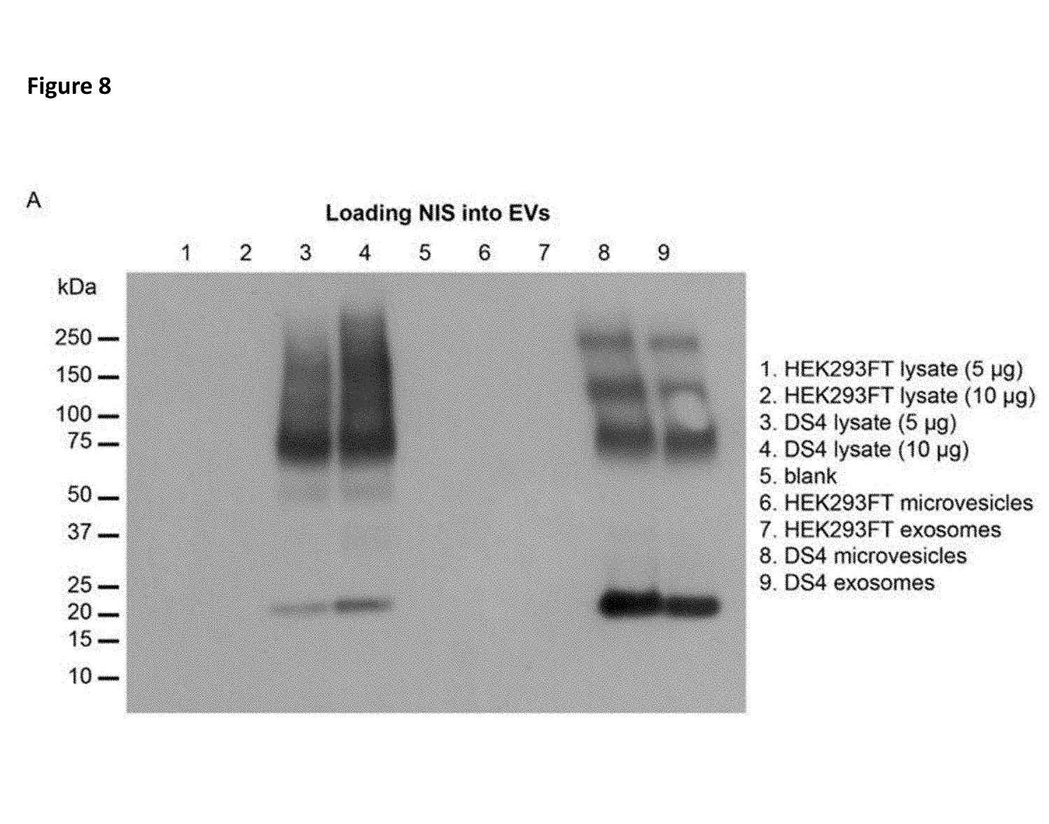

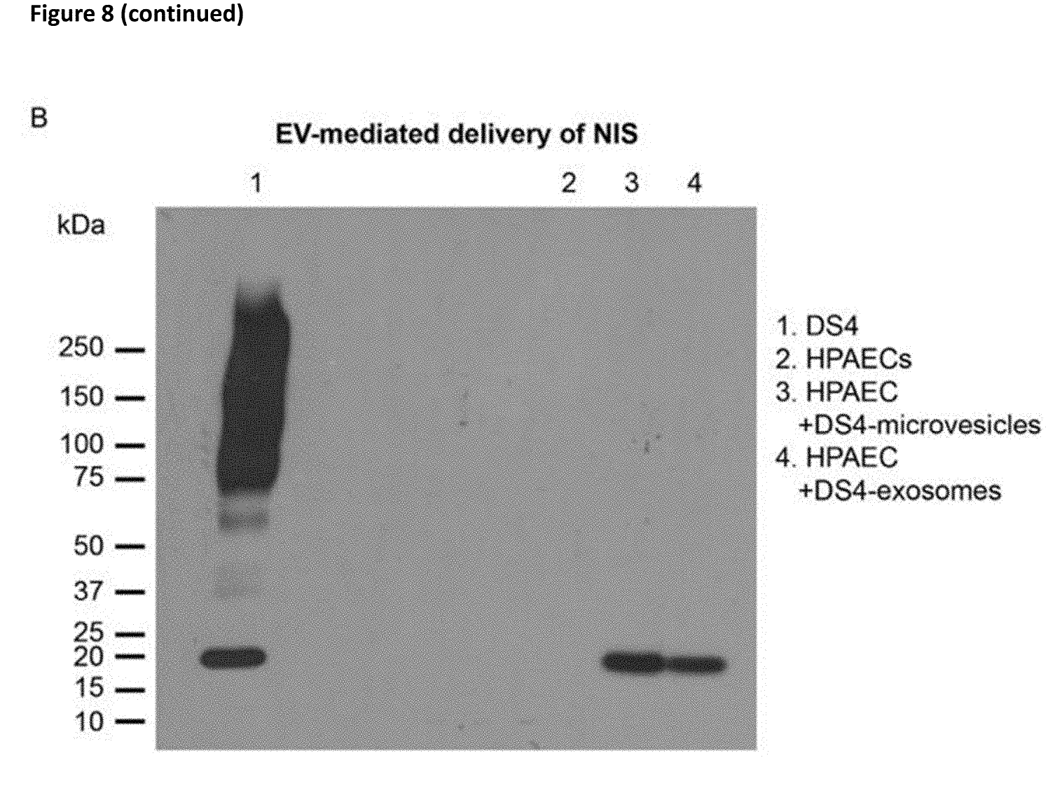

We first investigated whether NIS protein is incorporated into EVs. To this end, porcine NIS was stably expressed in EV-producing HEK293FT cells (via lentiviral transduction); this cell line was termed DS4. A FLAG affinity tag was genetically attached to C-terminus of NIS to facilitate detection in subsequent steps (yielding NIS-FLAG). EVs were harvested from such cells using a differential centrifugation protocol that selectively enriches for either microvesicles (which bud directly from the cell membrane) or exosomes (which originate from endosomal compartment) (See Appendix A for full methods). NIS content of DS4-derived EVs was evaluated by western blot. High levels of NIS were detected in both EV subpopulations, as well as in lysates of the parental DS4 cells (FIG. 8A). NIS was detected in several forms, including full length NIS (˜68 kDa), multiple glycoforms, and a small, ˜20 kDa band that corresponds to a well-characterized NIS-derived C-terminal fragment (7). The C-terminal fragment represents a cytosolic domain of NIS, which is cleaved once NIS reaches the cell surface but remains physically associated with the membrane-spanning portions of NIS to comprise a functional transporter. Note also that since we appended a FLAG tag to the C-terminus of NIS, our western blot would not detect the N-terminal NIS fragment. Thus, NIS is loaded into both microvesicles and exosomes, and at least some of this protein appears to be in the mature state normally found at the cell surface.

We next investigated whether NIS protein is delivered via EVs to recipient HPAECs. HPAECs were incubated with NIS-containing EVs for 16 hours, and then NIS content in recipient cells was evaluate by western blot. Most notably, NIS was detected in cells receiving either type of EVs (FIG. 8B). Interestingly, most NIS detected in recipient cells comprised the C-terminal fragment (noting again that our method would not detect the N-terminal fragment, which lacks a FLAG tag). Some larger NIS bands were detectable at larger exposure times (not shown), but the majority of the NIS delivered to recipient cells appeared to be the mature form of the protein.

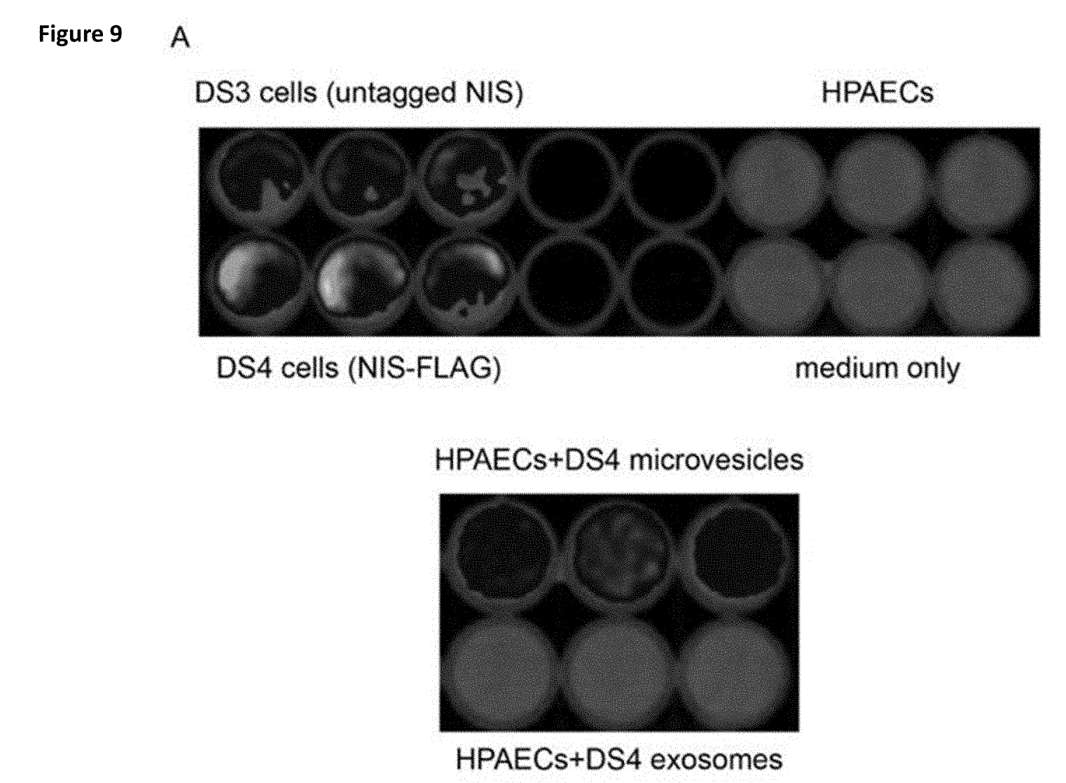

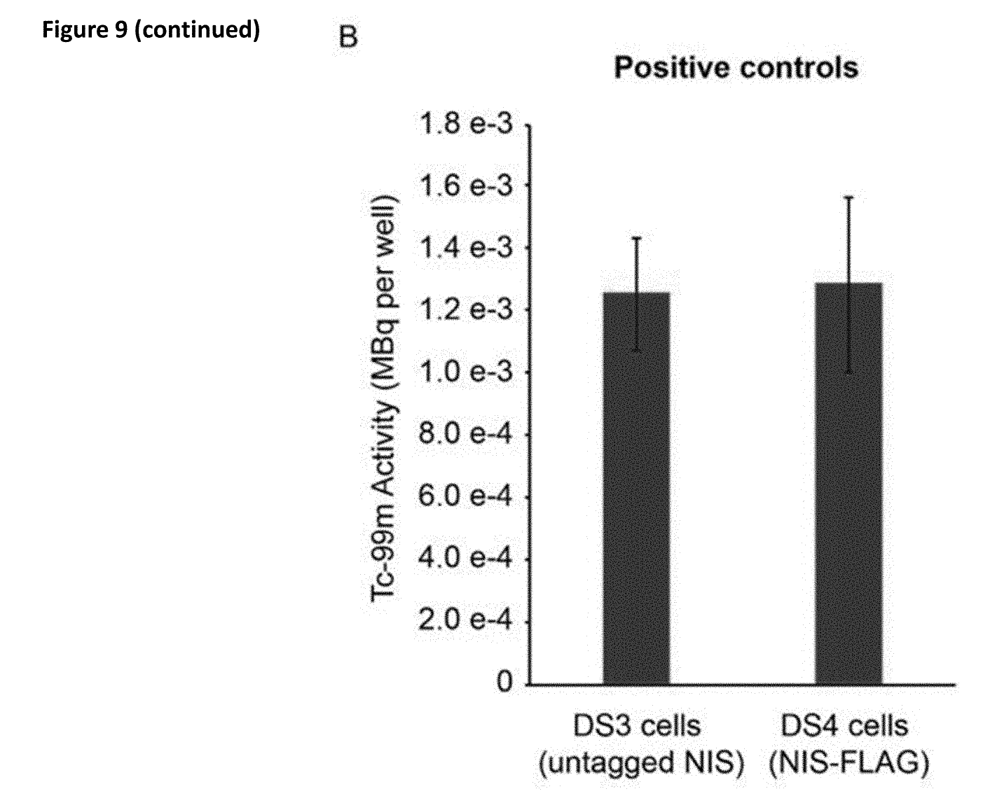

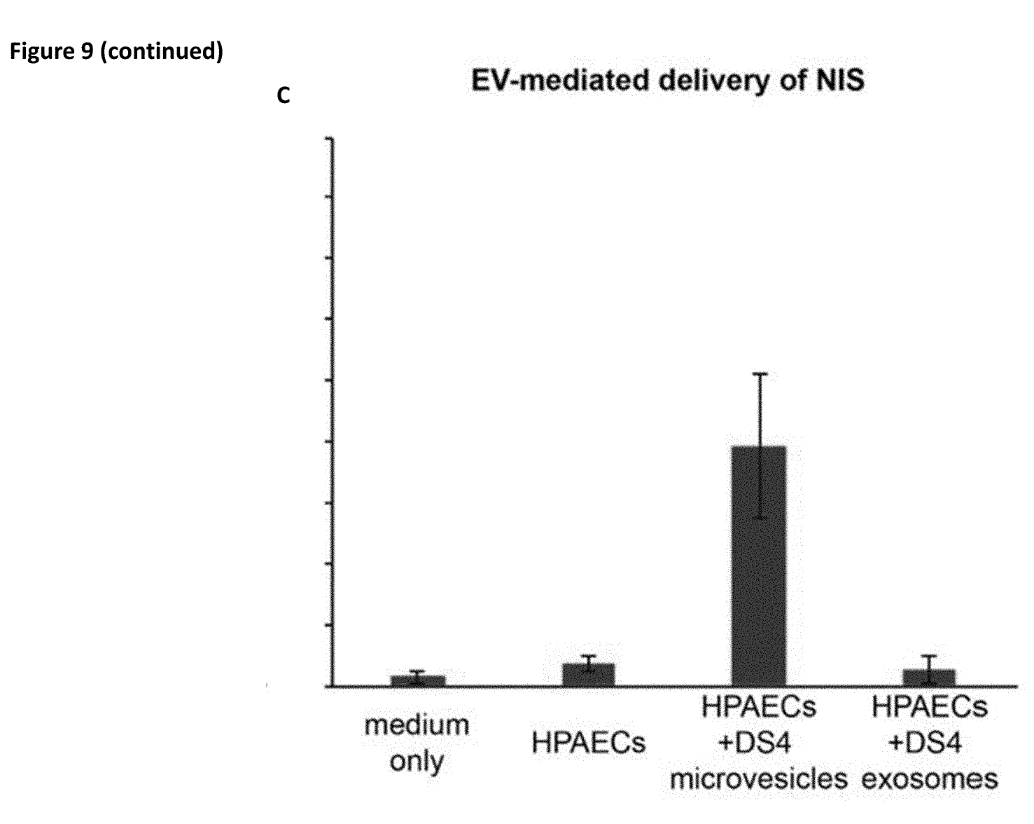

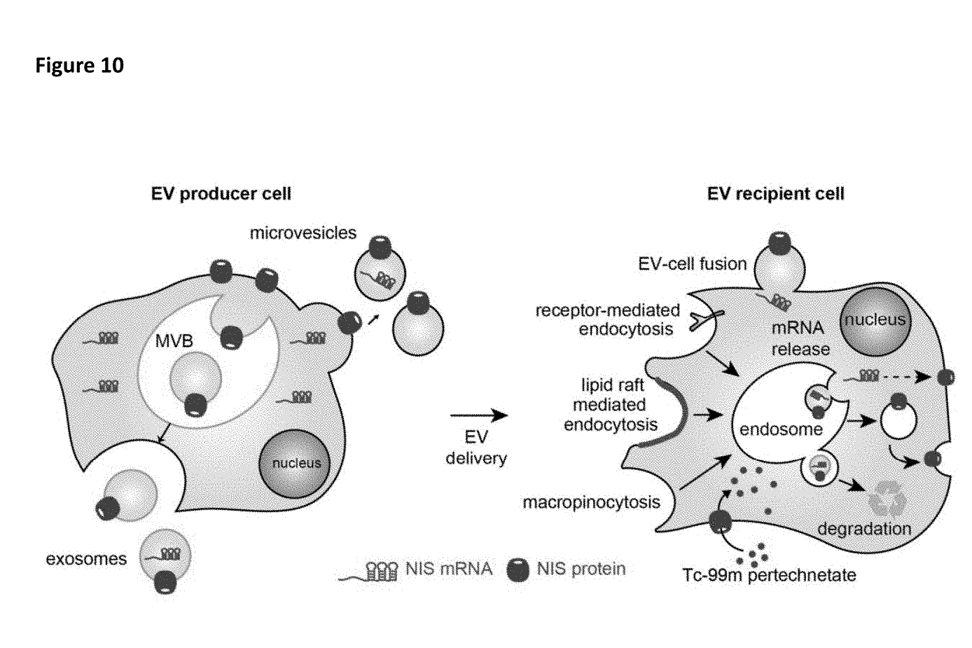

Finally, we investigated whether NIS protein delivered to recipient HPAECs is functional. As described above, we expect that functional delivery of NIS may require fusion between EV and HPAEC plasma membranes in a manner that preserves proper NIS orientation. To evaluate NIS function in recipient cells, HPAECs were treated with NIS EVs for 16 hours, and NIS-mediated uptake of the radioisotope Tc-99m pertechnetate was quantified via single photon emission computed tomography (SPECT). Parental NIS-expressing DS4 cells were evaluated as a positive control. Most notably, HPAECs treated with NIS microvesicles exhibited high levels of functional NIS activity, thus demonstrating, for the first time, that EVs mediate functional delivery of NIS to recipient cells (FIG. 9). Interestingly, HPAECs treated with NIS exosomes did not exhibit functional NIS activity (FIG. 9), despite the fact NIS protein uptake had been observed (FIG. 8B). Altogether, these data demonstrate that EV type impacts functional delivery of NIS to recipient cells. Whether these differences derive from incorporation of different forms of NIS, different methods and efficiencies of EV-plasma membrane fusion, or other factors remain to be elucidated (FIG. 10). Most importantly, these results demonstrate that EV-mediated delivery of functional membrane proteins, such as NIS, to HPAECs is a plausible and clinically attractive strategy.

Materials and Methods

Cell culture—HEK293FT cells (ThermoFisher Scientific) and HAPECs (gifted by Dr. Michael Passineau, Allegheny Health Network) were maintained at 37° C. in 5% CO₂. HEK293FT cells were cultured in DMEM supplemented with 10% FBS, 1% penicillin-streptomycin, and 4 mM L-glutamine Sublines generated from these cells (see Cell line generation, below) were cultured in the same way. HPAECs were cultured in Endothelial Cell Growth Medium (Cell Applications, Inc.).

Cell line generation—To package lentiviral vectors, HEK293FT cells were plated at ˜8×10⁵ cells/mL in 10 cm dishes (8 mL). 6-8 h later, when cells were ˜60-70% confluent and well attached to the plate, cells were transfected with 3 μg pMD2G, 8 μg pspax (gifted by William Miller, Northwestern University), 10 μg of viral vector (pGIPZ backbones) and 1 μg pDsRedExpress2 (Clontech). 12-14 h later, medium was changed. 28 h after the medium change, conditioned medium was harvested, cleared of cells by centrifugation at 500 g for 2 min, and filtered through a 0.4 μm pore filter (VWR). Cleared supernatant was then concentrated by ultracentrifugation at 24,200 rpm using an SW41 Ti rotor in an L-80 Optima XP ultracentrifuge for 90 min. The pellets were harvested and used to transduce ˜1.5×10⁵ HEK293FT cells. Transduced cells were selected for with puromycin. The resulting cell lines, DS3 and DS4, constitutively express NIS and NIS with a C-terminal FLAG tag, respectively.

EV isolation and characterization—EV-depleted medium was made by supplementing DMEM with 10% exosome-depleted FBS (Gibco), 1% penicillin-streptomycin, and 4 mM L-glutamine. For EV production, DS4 cells were incubated for 24 h before conditioned medium harvest, since EV concentration in conditioned medium was not observed to differ significantly at 24, 36 or 48 h of incubation, indicating that a steady state of EV production and uptake was reached by 24 h (Michelle Hung and Joshua Leonard, unpublished observations). EVs were isolated from conditioned medium by differential centrifugation at 4° C. Conditioned medium was centrifuged at 300 g for 10 mM (to remove cells) and 2,000 g for 20 min (to remove cell debris and apoptotic bodies); the supernatant was retrieved at each step for subsequent spins (8). From this clarified supernatant, microvesicles were pelleted at 15,000 g for 30 min; supernatant was again collected to pellet exosomes by ultracentrifugation at 26,500 rpm in an Optima XP ultracentrifuge (Beckman Coulter) with the SW41Ti rotor for 135 min at 4° C. Alternatively to centrifugation as utilized herein, EVs may be isolated and/or EV subpopulation may be enriched via affinity chromatography. (See Hung et al., “Enrichment of Extracellular Vesicle Subpopulations Via Affinity Chromography,” Mehtods Mol Biol. 2018:1740:109-124; the content of which is incorporated herein by reference in its entirety).

EV concentrations were profiled by NTA. A NanoSight LM10-HS (Malvern) with a laser wavelength of 405 nm and NanoSight NTA software v2.3. Videos were acquired at camera level 14. Samples were introduced manually. Three 30 s videos were analyzed per sample. EVs were diluted 1:50 or 1:100 in PBS to keep concentration between 2-9×10⁸ vesicles/mL. Vesicle concentration was defined as the mean of the concentrations determined from each of the 3 videos. Videos were analyzed at a detection threshold of 7. The blur, minimum track length, and minimum expected particle size were set automatically by the software.

EV delivery experiments—Recipient HPAECs were plated 24 h before EV delivery at ˜1.5×10⁵ cells/mL in 48 or 24 well plates (0.3 and 0.6 mL medium, respectively). EVs were counted by NanoSight, and equal numbers of vesicles were added to each well. 1×10¹⁰ vesicles were added per 5×10⁴ cells for NIS delivery. 16 hours after EV delivery, NIS levels in recipient cells were analyzed by western blot or SPECT. Positive control cells (DS3 and DS4 cells) were plated at ˜1.2×10⁵ cells/mL (in the same media volumes and well formats used for HPAECs) 40 h prior to lysis for western blot, or 16 h prior to SPECT.

Immunoblotting—For western blot analysis, EVs and cell lysates were heated in Laemmli buffer at 70° C. for 10 min. Protein concentration was measured by BCA assay (Pierce) for cell lysates, and several concentrations of each protein were loaded in each lane of a 4-15% gradient polyacrylamide gel (Bio-Rad). EV samples were normalized by vesicle count as determined by NanoSight. After transfer to a PVDF membrane (Bio-Rad) at 100 V for 45 min, membranes were blocked for 1 h in 5% milk at room temperature, and blotted with rabbit anti-FLAG antibody (Abcam ab1162) diluted 1:1000 and incubated overnight at 4° C. Primary antibodies were detected with horseradish peroxidase-conjugated goat-anti-rabbit immunoglobulin G secondary antibody (Thermo-Fisher Scientific).

SPECT—Cells were cultured in 48 well plates sawed in half lengthwise with a 300 series Dremel to produce a size compatible with the imager. Plates were sterilized by removing plastic fragments with an air hose, washing the wells with PBS, washing the plate with 70% ethanol, and allowing them to air dry. Cell culture and EV delivery was carried out as described above. All SPECT images were acquired at the Center for Advanced Molecular Imaging (CAMI) of the Chemistry of Life Processes Institute at Northwestern University (Evanston, Ill.), using microSPECT, MlLabs U-SPECT+/CT (MlLabs, Netherlands). For all samples, the radioisotope, technetium-99m, in the form of technetium pertechnetate (Na[^{99m}TcO₄]) at 0.300 mCi was diluted using DMEM for DS3 and DS4 cells and Endothelial Cell Growth Medium for HPAECs and added to each well/sample for 60 min with a heating pad around 37° C. The samples were washed twice with PBS to remove excess tracer prior to SPECT imaging. Wells with 300 μL of media per well were imaged (2 stacked, cut-well plates). The following parameters were used for data acquisition: 2 frames of 15 min each and General Purpose Rat and Mouse (GP-RM, 1.5 mm pinhole) collimator were used for SPECT, followed by CT (60 kV, 615 μA, 240 ms exposure, normal gantry speed, 360 projections). The data were reconstructed using OS-EM with 4 subsets, 6 iterations (24 Maximum Likelihood-Expectation Maximization, or ML-EM equivalent) and post filtered with 0.8 full width at half maximum (FWHM) filter. The voxel size was 0.4 mm³. CT was used for attenuation correction. A calibration factor of 585.7 MBq/(CPM*mL) was used to convert counts per minute to actual activity. Note, these parameters would be the same for in vivo imaging, i.e., this was an attempt to determine feasibility for future in vivo imaging. Images were analyzed using region of interest analysis on individual wells selecting all slices (i.e., to capture signal from each well in its entirety) using Amira 6.2.0 software.

### REFERENCES

1. Marcus M E & Leonard J N (2013) FedExosomes: Engineering Therapeutic Biological Nanoparticles that Truly Deliver. Pharmaceuticals (Basel) 6(5):659-680.
2. Alvarez-Erviti L, et al. (2011) Delivery of siRNA to the mouse brain by systemic injection of targeted exosomes. Nat Biotechnol 29(4):341-345.
3. Gyorgy B, Hung M E, Breakefield X O, & Leonard J N (2014) Therapeutic Applications of Extracellular Vesicles: Clinical Promise and Open Questions. Annual review of pharmacology and toxicology.
4. Samuel-Abraham S & Leonard J N (2010) Staying on message: design principles for controlling nonspecific responses to siRNA. FEBS J 277(23):4828-4836.
5. Vituret C, et al. (2016) Transfer of the Cystic Fibrosis Transmembrane Conductance Regulator to Human Cystic Fibrosis Cells Mediated by Extracellular Vesicles. Hum Gene Ther 27(2):166-183.
6. Penheiter A R, Russell S J, & Carlson S K (2012) The sodium iodide symporter (NIS) as an imaging reporter for gene, viral, and cell-based therapies. Curr Gene Ther 12(1):33-47.
7. Huc-Brandt S, et al. (2011) Characterisation of the purified human sodium/iodide symporter reveals that the protein is mainly present in a dimeric form and permits the detailed study of a native C-terminal fragment. Biochimica et biophysica acta 1808(1):65-77.
8. Jeppesen D K, et al. (2014) Comparative analysis of discrete exosome fractions obtained by differential centrifugation. Journal of extracellular vesicles 3:25011.

In the foregoing description, it will be readily apparent to one skilled in the art that varying substitutions and modifications may be made to the invention disclosed herein without departing from the scope and spirit of the invention. The invention illustratively described herein suitably may be practiced in the absence of any element or elements, limitation or limitations which is not specifically disclosed herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention. Thus, it should be understood that although the present invention has been illustrated by specific embodiments and optional features, modification and/or variation of the concepts herein disclosed may be resorted to by those skilled in the art, and that such modifications and variations are considered to be within the scope of this invention.

Citations to a number of patent and non-patent references may be made herein. The cited references are incorporated by reference herein in their entireties. In the event that there is an inconsistency between a definition of a term in the specification as compared to a definition of the term in a cited reference, the term should be interpreted based on the definition in the specification.

We claim:

1. Extracellular vesicles comprising a sodium iodide symporter (NIS) or nucleic acid encoding the sodium iodide symporter (NIS) protein.

2. The extracellular vesicles of claim 1, wherein the extracellular vesicles are exosomes or microvesicles.

3. The extracellular vesicles of claim 1, wherein the nucleic acid is mRNA encoding the NIS protein.

4. The extracellular vesicles of claim 1, further comprising a surface ligand that binds to receptor on a recipient cell.

5. The extracellular vesicles of claim 1, further comprising a therapeutic agent for delivery to a recipient cell.

6. The extracellular vesicles of claim 1, wherein the NIS comprises the amino acid sequence of SEQ ID NO:1 or an amino acid sequence having at least about 80% sequence identity to SEQ ID NO:1.

7. The extracellular vesicles of claim 1, further comprising a cationic polymer on their surfaces.

8. A method comprising: (a) contacting extracellular vesicles with recipient cells, the extracellular vesicles comprising a heterologous cell membrane transporter protein or nucleic acid encoding the heterologous cell membrane transporter protein; (b) contacting the recipient cells with a labeled substrate for the heterologous cell membrane transporter protein; and (c) detecting uptake of the labeled substrate in the recipient cells.

9. The method of claim 8, wherein the heterologous cell membrane transporter protein is the sodium iodide symporter (NIS) protein.

10. The method of claim 9, wherein the labeled substrate is radioactive iodide or radioactive technetate.

11. The method of claim 10, wherein the radioactive technetate is ^{99m}Tc-pertechnetate).

12. The method of claim 8, wherein prior to contacting the extracellular vesicles with the recipient cells, the extracellular vesicles are treated with a cationic polymer.

13. The method of claim 8, wherein the recipient cells are contacted with the extracellular vesicles under centrifugal force

14. A method for preparing the extracellular vesicles of claim 1, the method comprising expressing in an EV-producing cell line a sodium iodide symporter (NIS) protein and isolating extracellular vesicles comprising the NIS protein or nucleic acid encoding the NIS.

15. The method of claim 14, wherein expressing the NIS protein in the EV-producing cell line comprises introducing into the EV-producing cell line a heterologous nucleic acid that encodes the NIS protein.

16. A recombinant EV-producing cell line into which a heterologous nucleic acid that encodes a sodium iodide symporter (NIS) protein has been introduced.


SEQUENCE LISTING (1 sequence)

SEQ ID NO: 1 - protein, 643 aa, Homo sapiens
MEAVETGERPTFGAWDYGVFALMLLVSTGIGLWVGLARGGQRSAEDFFTGGRRLAALPVGLSLSASFMSAVQVLGVPSEAYRYGLKFLWMCLGQLLNSVLTALLFMPVFYRLGLTSTYEYLEMRFSRAVRLCGTLQYIVATMLYTGIVIYAPALILNQVTGLDIWASLLSTGIICTFYTAVGGMKAVVWTDVFQVVVMLSGFWVVLARGVMLVGGPRQVLTLAQNHSRINLMDFNPDPRSRYTFWTFVVGGTLVWLSMYGVNQAQVQRYVACRTEKQAKLALLINQVGLFLIVSSAACCGIVMFVFYTDCDPLLLGRISAPDQYMPLLVLDIFEDLPGVPGLFLACAYSGTLSTASTSINAMAAVTVEDLIKPRLRSLAPRKLVIISKGLSLIYGSACLTVAALSSLLGGGVLQGSFTVMGVISGPLLGAFILGMFLPACNTPGVLAGLGAGLALSLWVALGATLYPPSEQTMRVLPSSAARCVALSVNASGLLDPALLPANDSSRAPSSGMDASRPALADSFYAISYLYYGALGTLTTVLCGALISCLTGPTKRSTLAPGLLWWDLARQTASVAPKEEVAILDDNLVKGPEELPTGNKKPPGFLPTNEDRLFFLGQKELEGAGSWTPCVGHDGGRDQQETNL